(19) United States
(12) Patent Application Publication
(10) Pub. No.: US 2005/0165459 A1
(43) Pub. Date: Jul. 28, 2005

(54) Apparatus for diminishing pain and/or hemorrhage caused by a therapeutic injection, body tissue sampling or injury

(76) Inventor: George Gary Coulter, Nashville, IN (US)

Correspondence Address: WILSON SONSINI GOODRICH & ROSATI, 650 PAGE MILL ROAD, PALO ALTO, CA, 943041050, US

(21) Appl. No.: 11/022,269

(22) Filed: Dec. 22, 2004

Related U.S. Application Data
(63) Continuation-in-part of application No. 10/746,685, filed on Dec. 29, 2003.

(51) Int. Cl. A61N 1/18

(52) U.S. Cl. 607/46

(57) ABSTRACT

A device couples the use of Trans Epithelial Nerve Stimulation with the administration of a therapeutic injection, such as an immunization, or tissue sampling procedure such as deriving a blood sample. By such an arrangement, the discomfort associated with these procedures may be considerably reduced or eliminated, thereby improving compliance with a range of medical procedures.

### CROSS-REFERENCE

This is a continuation-in-part application of a co-pending U.S. Patent Application Publ. No. 2004/0015188, filed Dec. 29, 2003, the disclosure of which is incorporated herein by reference.

### BACKGROUND OF THE INVENTION

Technology developments in effecting a therapeutic injection, such as an immunization, or in sampling a body tissue, such as blood, have predominantly been orientated around the avoidance of cross contamination, accuracy of injection, or avoidance of operator injury. Some diminution of pain of the recipient has often been a secondary consideration and has proved a somewhat elusive goal. This is due, at least in part, to some of the factors defining the likelihood and extent of pain being related to the nature of substance to be injected, site and depth of injection and the psychological expectation of pain by the patient. For the most part, the common injections performed are regarded as inducing only minor and transient pain or discomfort. However, despite the modest level of discomfort associated with many common injections, needle phobia and the discomfort of injection remain as significant barriers to many therapies. For example, regimens for diabetes control are sometimes not adhered to because of the discomfort inherent in blood glucose testing and or insulin injection.

Nature builds in a great deal of redundancy in all its systems including to communication. For example in written communication the well known sentence “if u cn rd the msg . . . .” clearly indicates the redundancy of information communicated in a correctly spelled sentence. The same strategy of reliability through the use of redundancy is true of the various biological systems of the body with generally overcapacity of each system and adjunctive, nonessential activity in each system. Thus, the same message sent by multiple routes (nerve fibers) can accommodate some noise in each route but the combined central message arrives adequate for clear interpretation. In the peripheral nervous system this generally ensures more certain communication, in the same way as “packeting” of information within a telephone system, with multiple routes taken by the packets ensuring more noise-free and certain delivery of the message. With approximately 670 neurites or more per square millimeter of the skin of the finger tips, yet a two point discrimination ability of around two millimeters, there appears to be massive built-in redundancy. Translating the implications of these insights into an effort of controlling the peripheral nervous system suggests that completely blocking the nervous impulses generated on tissue injury in a given area, such as the site of injection, is unlikely to be fully effective, unless exceedingly strong means are employed.

Devices making use of TENS (trans-epithelial nerve stimulation) for the reduction of pain are known. The current embodiments of TENS devices are not optimized for the anatomical location of nerves within the skin or the local intensity of exposure required for significant local blockade of pain. Efforts have been conducted in the general development of TENS elucidating various parameters of pulse size, pulse width, form, number and spacing of pulses in pulse trains and electrode contact design. At best, these efforts allow for optimizing this weak form of pain control for deeper tissues, and for the most part they have a poor record of success. Many devices are in the public domain and are generally used in the management of chronic pain. In the case where TENS has been proposed for acute use, it is noted that the application is clearly intended as an alternative or adjunct to the use of a pharmacological anesthetic when a surgical intervention is envisaged, and is intended to operate for some minutes, as described in U.S. Pat. Nos. 6,351,674, 4,924,880, and 5,052,391. With the exception of U.S. Patent Application Publ. No. 2004/0015188 (U.S. Ser. No. 10/195,171), no TENS device has been considered explicitly for the avoidance of the pain expected and induced by a short procedure such as an injection (where indeed the agent being injected may be a local anesthetic). Equally limited success has been seen with manipulating the mechanics of injection, such as needleless injector technology, or refinement of the needle systems into automatic or triggered devices. The principal strategy employed by these designs is to minimize trauma and or “catch the patient unaware”. These have been proposed for either injection or for the collection of small blood samples, for example, in the assessment of diabetes, as described in U.S. Pat. Nos. 6,135,979, 6,102,896, 6,083,197, 5,993,412, and 5,746,714.

Despite these above-described efforts, pain on therapeutic injection or sampling remains an ongoing and potentially avoidable issue. In the case of current TENS devices of particular note, the various designs of the current-applying electrodes attempt to maximize general contact area and conductivity, thereby improving effectiveness of current transfer to the body part. This may be desirable where a general counter irritation, muscle stimulation or other deep tissue stimulation is a targeted object of TENS application. However, where a specific localized superficial blockade is desired and includes targeting of intra epithelial and dermal nerves, the usual large area electrode arrangement would be inappropriate, as good control of the anatomy of current flow is lost.

Partial alleviation of these concerns and effects can be achieved by the use of the device described in U.S. Patent Application Publ. No. 2004/0015188. However, there is a further need in the art for the reduction of pain associated with injections and body tissue sampling.

### SUMMARY OF THE INVENTION

The present invention provides an apparatus for reducing pain or hemorrhage. The apparatus comprises a current generating device configured to generate an electrical output Trans Epithelial Nerve Stimulating (TENS) current. The apparatus further comprises an array of electrodes electrically coupled to the current generating device and configured to be placed around an injection location on the skin of a patient. Each of the electrodes has a surface area of less than five square millimeters.

The present invention further provides an apparatus for reducing pain or hemorrhage at an injection location on the skin of a patient, the device comprising a current generating device configured to generate first and second types of electrical output Trans Epithelial Nerve Stimulating (TENS) current. The first type of TENS current has a frequency greater than about 15 Hz, while the second type of TENS current has a frequency less than about 15 Hz. The device further comprises an array of electrodes electrically coupled to the current generating device and configured to be placed around the location on the skin of the patient. The electrodes include a first group and a second group, the first group being configured to be disposed closer than the second group to the injection location on the skin of the patient. The device further comprises a current applying device configured to apply the first type of TENS current to the first group of electrodes and apply the second type of TENS current to the second group of electrodes.

The present invention further provides a method of reducing pain or hemorrhage of a therapeutic injection or a body tissue sampling. The method comprises the steps of placing an array of electrodes on the skin of a patient, applying electrical output Trans Epithelial Nerve Stimulating (TENS) current to the electrodes, and breaching the skin of the patient at a location within the array of electrodes during the applying step.

The present invention further provides a method of reducing pain or hemorrhage. The method comprises the steps of placing an array of electrodes into the wound of a patient, and applying electrical current to the array of electrodes.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a block diagram of one embodiment of an apparatus of the present invention for reducing pain or hemorrhage;

FIG. 2 is a schematic block diagram of one embodiment of the transformers and optoisolators of FIG. 1;

FIG. 3 is a schematic diagram of one embodiment of the pulse width control circuit of FIG. 1;

FIG. 4 is a schematic diagram of one embodiment of one of the gain control circuits of FIG. 1;

FIG. 5 is a plan view of one embodiment of the electrodes of FIG. 1;

FIG. 6 is a plan view of another embodiment of the electrodes of FIG. 1; and

FIG. 7 is a listing of a portion of code that may be used in conjunction with the apparatus of FIG. 1.

Corresponding reference characters indicate corresponding parts throughout the several views. Although the exemplification set out herein illustrates an embodiment of the invention, the embodiment disclosed below is not intended to be exhaustive or to be construed as limiting the scope of the invention to the precise form disclosed.

### DETAILED DESCRIPTION OF THE INVENTION

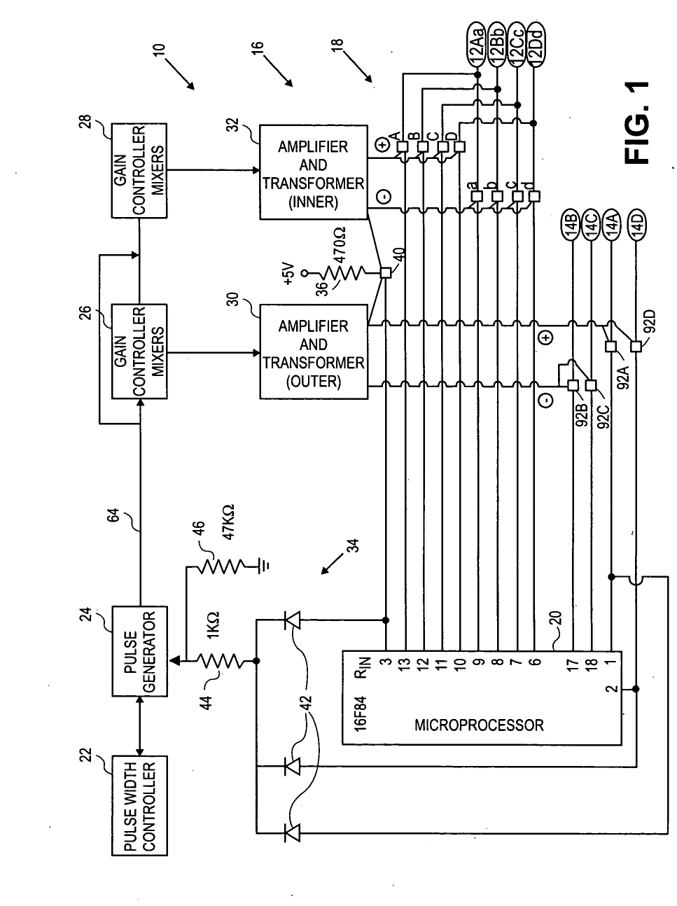

Referring now to the drawings, and particularly to FIG. 1, there is shown one embodiment of an apparatus 10 of the present invention for reducing pain or hemorrhage. Apparatus 10 may be used in conjunction with, or as an enhancement of, the invention disclosed in U.S. Patent Application Publ. No. 2004/0015188. Apparatus 10 includes an array or group of first electrodes 12Aa, 12Bb, 12Cc, 12Dd, and an array or group of second electrodes 14A, 14b, 14c, 14D for being placed upon the outer surface of the epidermis of a patient. As described in more detail below, electrodes 12 may be placed on the patient's skin closer to the injection site or injury than are electrodes 14. Electrodes 14 may substantially surround or encircle electrodes 12. Thus, electrodes 12 may be referred to herein as “inner” electrodes, and electrodes 14 may be referred to herein as “outer” electrodes. Apparatus 10 also includes a microcomputer-controlled, TENS current generating circuit 16 having an electronic switch matrix 18 for sequentially applying TENS current signals to electrodes 12, 14. Thus, circuit 16 is electrically coupled to electrodes 12, 14. Circuit 16 also includes a microcontroller 20, a pulse width controller 22, a pulse generator 24, gain controllers 26, 28, and amplifier and transformer sections 30, 32.

Microcontroller 20 may be in the form of any of a number of microprocessor models, such as those of the BASIC STAMP series produced by Parallax, Inc. of Rocklin, Calif., or of the PIC micro series. In one embodiment, microcontroller 20 may be embodied by the PIC 16F84 produced by Microchip Technology Inc. of Chandler, Ariz., or its one time programmable equivalent.

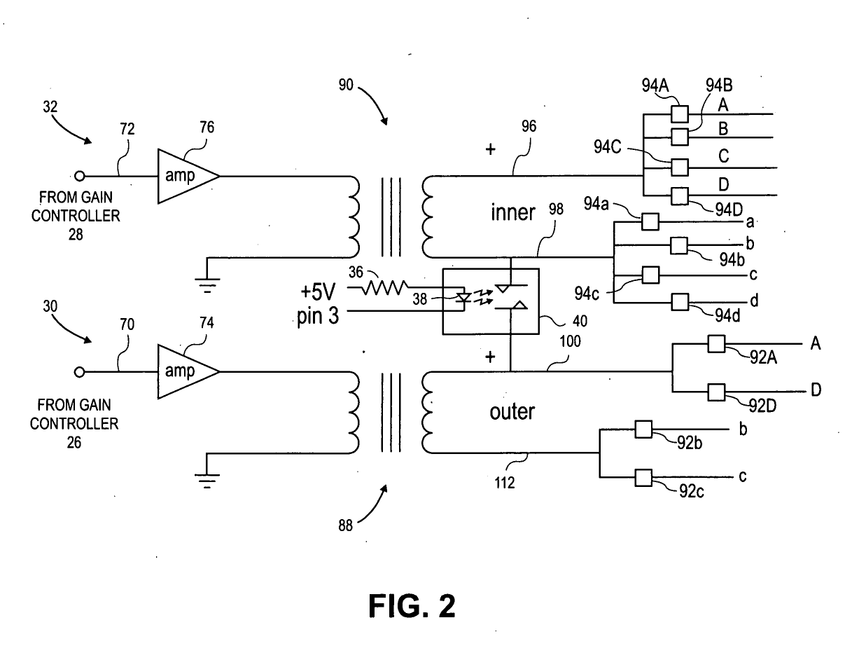

Pulse generator 24 may be in the form of a standard 555 timing chip for generating a pulse signal. Alternatively, it is possible for the pulse signal to be generated by microprocessor 20 without the need for a separate pulse generator 24. Pulse generator 24 may be triggered by outputs from microprocessor 20 via a trigger mixer circuit 34. More particularly, pulse generator 24 may be triggered when one or more suitably connected microprocessor outputs (e.g., pins 1-3 in the embodiment shown in FIG. 1) are low, i.e., transmit zero voltage. Any one of the microprocessor outputs being in a high state prevents triggering. Pin 3 of the PIC 16F84 is an open collector output and therefore may require a pull up resistor 36 in order to go high. Pin 3 may be connected to a five volt voltage supply via resistor 36 and through a diode 38 (FIG. 2) of an optoisolator 40. Optoisolator 40, like the other optoisolators of circuit 16, may be a model NTE 3047 optoisolator sold by NTE Electronics, Inc. of Bloomfield, N.J. The outputs of pins 1-3 may be fed via signal diodes 42 into a resistor ladder including resistors 44, 46, and then to a trigger pin 2 of a 555 timer chip 24.

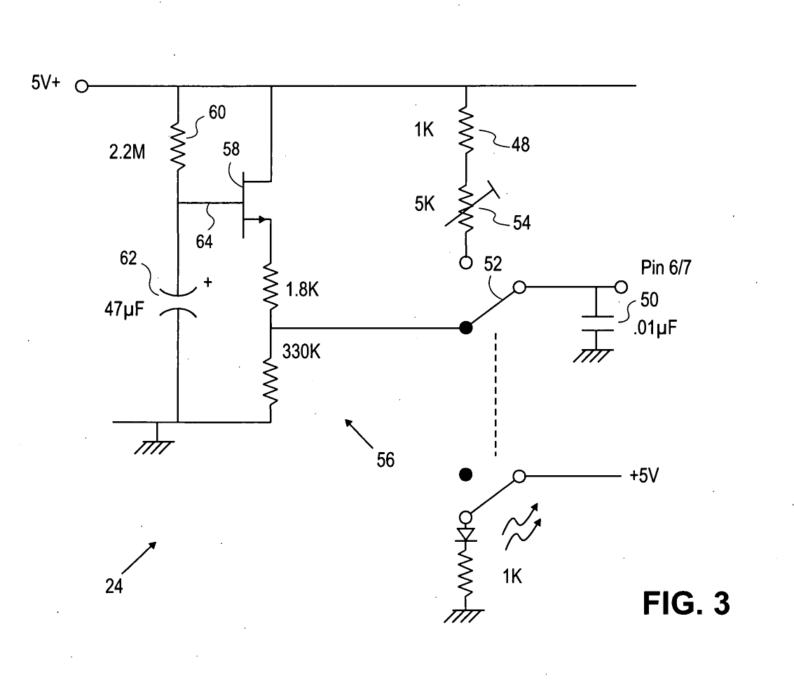

The widths of the pulses generated by the 555 timer chip 24 may be controlled by a standard combination of resistors 48 and 54 (FIG. 3) and a capacitor 50 (0.01 microfarads) of pulse width controller 22. A two way switch 52 connects pin 6 or 7 of 555 timer 24 to either a standard resistor-capacitor arrangement 48, 52 and 54, or to a circuit 56 which uses the variable transconductance of a FET 58 modulated by a combination of a resistor 60 and a capacitor 62 connected to gate 64 of FET 58. Thus, a relatively large pulse width may be initially provided, and then the pulse width may decline to a shorter length over a few seconds.

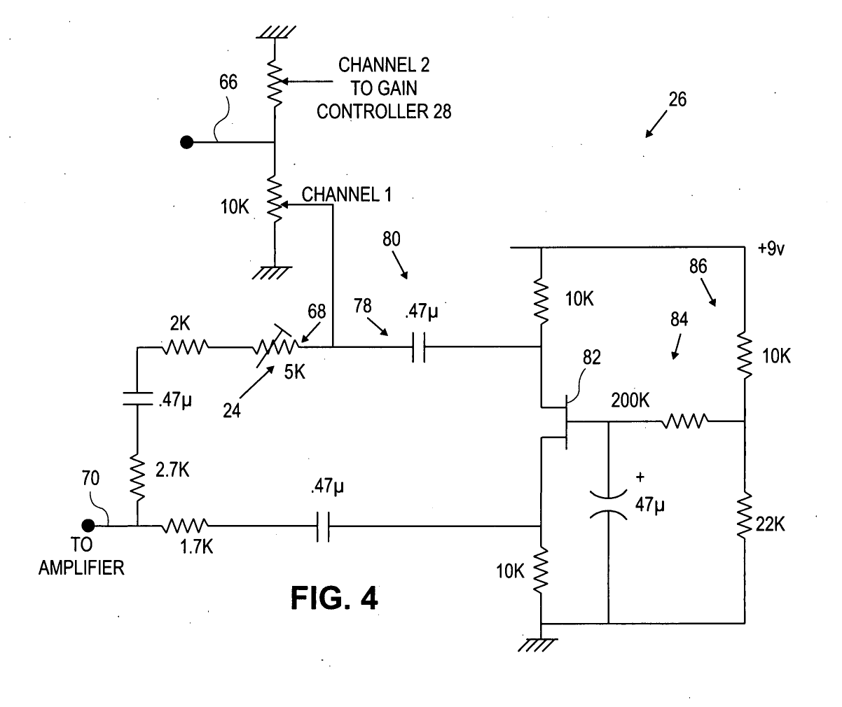

An output 66 of timer 24 may be split between gain control circuits 26, 28 as shown in FIG. 4. Each of channels 1 and 2 may have a bypass route 68 through a resistance to a respective input 70, 72 of a respective channel amplifier 74, 76, as shown in FIG. 2. Each of the channels 1 and 2 may have a route 78 through an FET circuit 80. The transconductance of a FET 82 may be controlled by an RC circuit 84 and a voltage divider circuit 86, resulting in an initial signal at amplifier input 70 that approximately doubles in strength, i.e., magnitude, over a time period of approximately between five and eight seconds. Channels 1 and 2 may have different time constants (governed by the RC combinations), thereby allowing a gentle and comfortable buildup of strong signals eventually reaching the patient. In one embodiment, both of FETs 58 and 82 are in the form of a MPF-102 model FET.

As shown in FIG. 2, the two output transformers 88, 90 may be joined by optoisolator 40. Within switch matrix 18, the outputs of transformers 88, 90 may be joined through optothyristors 92A, 92b, 92c, 92D, 94A-D and 94a-d to the electrode elements 12, 14, as shown in FIG. 1. Optothyristors have the advantage of being able to switch high levels of voltage at high frequency, with current flowing in either direction. The two-way switching provided by such optothyristors may electrically isolate electrodes 12, 14, and allow either positive or negative voltage to be applied to each of electrodes 12, 14. Thus, the optothyristors may be referred to herein as “optoisolators”. Because the signal is unipolar (the output of the transformer may be beneficially protected with a kick back diode), it is possible to control the electrical status and polarity of each electrode element 12, 14 individually. A positive terminal 96 of transformer 90 may be connected via optothyristors 94A-D to the inner set of electrode elements 12Aa, 12Bb, 12Cc, 12Dd. A negative terminal 98 may be connected to the same four electrode elements 12 by another set of optothyristors 94a-d. To signify whether the element is connected to a positive terminal or a negative terminal of its corresponding transformer, the electrode elements are herein labeled a to d (lower case) in the case of a negative terminal, and are labeled A to D (upper case) for a positive terminal. However, it is possible for optoisolators 94a-d to be connected to a positive terminal 100 of transformer 88 by activating optoisolator 40 via pin 3 of processor 20. Similarly, it is possible for optoisolators 92A, 92D to be connected to a negative terminal 98 of transformer 90 by activating optoisolator 40. Thus, a negative voltage may be applied to electrodes 14A, 14D.

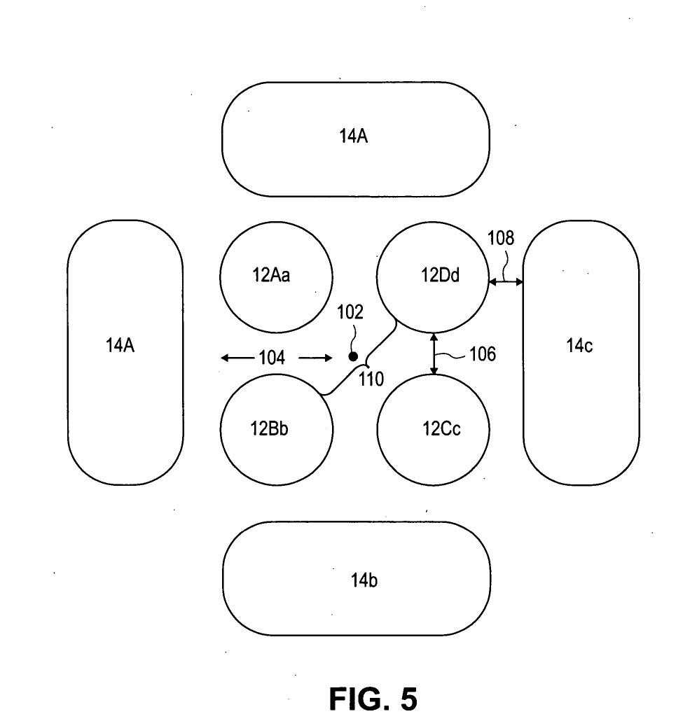

FIG. 5 illustrates one embodiment of a plan layout of an electrode element matrix including sets of electrodes 12, 14. It should be noted that FIG. 5 is not necessarily drawn to scale, but rather illustrates the general positional relationships of the patient contact surfaces of the electrode elements 12, 14 of the matrix. Electrodes 14 are shown as being larger and of the same number as electrodes 12. However, it is possible for electrodes 14 to be the same size or smaller than electrodes 12. Further, it is possible for the number of electrodes 14 to be greater or less than the number of electrodes 12.

The electrically conducting material of electrodes 12, 14 may be metal or a conducting gel backed onto a suitable insulating material and may be disposable or capable of being recycled and reused. The patient side of electrodes 12, 14 may be provided with a sticky or adherent nature so that the electrode may be adhered to the patient's skin during use. Each of first electrodes 12Aa, 12Bb, 12Cc, 12Dd, and second electrodes 14A, 14b, 14c, 14D may be electrically isolated from all other ones of the electrodes.

The electrode arrays of FIG. 5 illustrate one of many possible tessellated arrangements of the individual electrode contact surface shapes. The dorsum of each of the electrodes may include an insulated wiring surface that connects through to the ventral surface, i.e., the surface of the electrode that touches the skin and that is visible in FIG. 5. Thus, suitable electrical connections may be made and routed via a suitable connector and then to TENS generating circuit 16.

Electrodes 12, 14 may be arranged around a point 102 where skin penetration or injury occurs. Point 102 may be surrounded or circumscribed by inner electrodes 12, and electrodes 12 may be disposed proximal to point 102. In the illustrated embodiment, there are four electrodes 12 that may each be less than 5 and preferably less than 4 square millimeters in area. In one embodiment, each of inner electrodes 12 has a width 104 or diameter of approximately 0.8 millimeter and an area of approximately 0.5 square millimeters. It is possible for each of electrodes 12 to be no more than two millimeters away from an adjacent electrode 12. In the embodiment shown in FIG. 5, distances 106 between adjacent inner electrodes 12 and distances 108 between adjacent electrodes 12, 14 may each be less than 1.0 millimeter. In one embodiment, distances 106, 108 are less than 0.5 millimeter. A distance 110 between electrodes 12 on opposite sides of point 102 may be less than 1.5 millimeter. In one embodiment, distance 110 is less than 1.0 millimeter. Thus, point 102 may be less than 0.75 millimeter from an adjacent electrode 12, and, in one embodiment, may be less than 0.50 millimeter away. In applications where distance 110 needs to be enlarged in order to accommodate a needle diameter, additional inner electrode elements may be provided, thereby enhancing flexibility in programming and reliability.

By activating the optoisolators in pairs, connections between the various electrode elements and the poles of the output transformers may be made. A ring of discharge pathways through the patient's skin may be sequentially created by activating electrode element pairs around point 102, thereby providing good coverage of the sensory nerves in the area. The signals applied to electrodes 12 may effectively encircle injection point 102 and may have a plurality of pulses of varying power and varying duration. The voltage signals applied to electrodes 12, 14 may cause a current through the patient's skin. The voltage signals applied to inner electrodes 12 in particular may depolarize the nerves within the skin to thereby suppress pain sensitivity. Nerves are naturally electrically polarized, which may facilitate the transmission of pain signals. For example, the inside of a nerve may be at a potential of −70 millivolt while the outer surface of the nerve is at a positive potential. Thus, a negative voltage applied to the outside of the nerve may depolarize the nerve and inhibit its ability to transmit a pain signal. Moreover, a positive voltage applied to the outside of the nerve may hyper-polarize the nerve and also inhibit its ability to transmit a pain signal. The voltage applied to the inner electrodes 12 may affect the C fibers in the skin to thereby reduce the visceral sensation of pain.

The voltage applied to outer electrode elements 14A, 14b, 14c, 14D may provide electrical background noise that tends to obscure any residual pain signals. Thus, the outer electrodes may mask the timing of the injection. The voltage applied to the outer electrodes 14 may affect the A-delta fibers in the skin to thereby reduce the sensation of pain related to sharpness. The voltages applied to electrodes 12 and 14 may exceed 300 volts, such as 360 to 500 volts. Alternatively, voltages of up to approximately 1000 volts may be applied to electrodes 12 and 14.

It is not necessary for outer electrode elements 14A, 14b, 14c, 14D to be capable of connection to both positive and negative transformer terminals. Rather, discharge is sufficient, for example, if one of electrodes 14A, 14D is positive and one of electrodes 14b, 14c is negative. Further, there may be more flexibility in the shape and size of outer electrode elements 14A, 14b, 14c, 14D than in the shape and size of inner electrode elements 12Aa, 12Bb, 12Cc, 12Dd. Outer electrode elements 14A, 14b, 14c, 14D are shown as being of greater size than inner electrode elements 12Aa, 12Bb, 12Cc, 12Dd. However, in another embodiment (not shown) the outer electrode elements are of approximately the same size as the inner electrode elements and are joined together in pairs.

With the electrode elements arranged as shown in the drawings, optoisolator 40 may join the two output transformers 88, 90. In another embodiment, it is possible for a connection between positive terminal 96 of inner transformer 90 and a negative terminal 112 of outer transformer 88 to be made with an optoisolator. In this arrangement, an inner element may be activated in association with an outer element and the inner element may be the negative pole when the optoisolator is on. This enhances the ability to maintain a depolarizing current with the inner area covered by electrodes 12Aa, 12Bb, 12Cc, 12Dd. Alternatively, reversing the connections between the output transformers provides an opportunity to make all outer electrode elements including 14A and 14D serve as negative poles.

A pulse voltage of up to 1000 volts may be provided at the output side of the output transformers 88, 90. In one embodiment, transformers 88, 90 have turns ratios of 1:44. The impedance of primary coils 114, 116 may define the need for the power rating of the output circuitry in gain controllers 26, 28. A higher primary impedance may allow for lower power output components in gain controllers 26, 28.

TENS generating circuit 16 may be housed as described in U.S. Patent Application Publ. No. 2004/0015188. Alternatively, circuit 16 may be housed in a suitable container such that either the patient or administering caregiver may control the various settings and levels. In this case, the output of circuit 16 may be via a multi-cored cable to suitable electrodes.

Both inner electrodes 12 and outer electrodes 14 may be energized in random order. However, it is possible for either or both of the inner electrode array and the outer electrode array to be energized in a clockwise or counterclockwise encircling pattern. For example, the inner electrodes may be energized in a counterclockwise pattern with a repeating sequence of 12Aa, 12Bb, 12Cc, 12Dd, 12Aa, . . . , etc. Outer electrodes may also be energized in a counterclockwise pattern with a repeating sequence of 14A, 14b, 14c, 14D, 14A, etc. Moreover, it is possible for one or more inner electrodes, one or more outer electrodes, or both an inner electrode and an outer electrode to be energized at a same moment in time. The encircling pattern of the first group of electrodes may be different from the encircling pattern of the second group of electrodes.

Generally, inner electrodes 12 may be individually energized with a frequency of greater than 15 Hz, and outer electrodes 14 may be individually energized with a frequency of less than 15 Hz. More particularly, inner electrodes 12 may be individually energized with a frequency approximately between 25 and 200 Hz. In one embodiment, inner electrodes 12 are energized with a frequency of approximately between 30 and 150 Hz. Outer electrodes 14 may be individually energized with a frequency of less than approximately 10 Hz. In one embodiment, individual outer electrodes 14 may be energized with a frequency of approximately between 3 and 4 Hz. Inner electrodes 12 and outer electrodes 14 may be independently controlled. Thus, circuit 16 may generate one type of TENS current for inner electrodes 12 and another type of TENS current for outer electrodes 14.

Although representative frequencies are provided herein, it is to be understood that the signals may be applied to both inner electrodes 12 and outer electrodes 14 with a degree of randomness and with irregular time intervals between pulses. Thus, the signals applied to electrodes 12, 14 may not have consistent frequencies or consistent time intervals between successive pulses.

A time duration of the application of TENS current to electrodes 12, 14 may be less than 30 seconds. In one embodiment, a time duration of the application of TENS current to electrodes 12, 14 may be less than 10 seconds. The patient's skin may be breached, such as by an injection needle, during the period of application of TENS current. The skin may be breached at a location, such as injection site 102, which is closer to electrodes 12 than to electrodes 14.

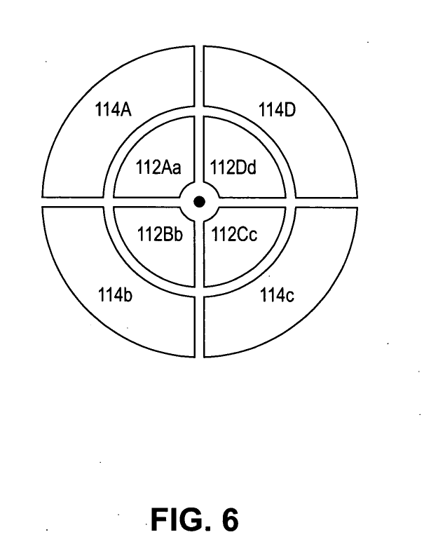

FIG. 6 illustrates another embodiment of electrodes that may be used in conjunction with the present invention. Inner electrodes 112Aa, 112Bb, 112Cc, 112Dd and outer electrodes 114A, 114b, 114c and 114D are arranged in a generally circular pattern around an injection site 202 to thereby increase the amount of surface area covered by the electrodes. The increase in surface area covered by the electrodes may be useful in inhibiting hemorrhaging. FIG. 6 is not drawn to scale, and thus is not directly comparable to FIG. 5 in terms of sizes of the electrodes.

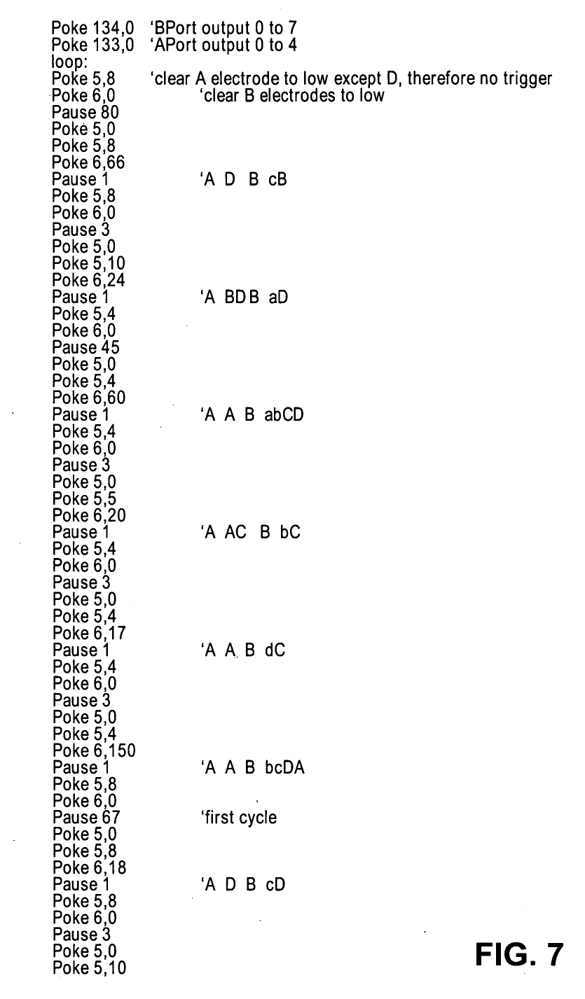

FIG. 7 provides a sample of computer code suitable for use when processor 20 is in the form of a PIC 16F84. The code illustrates the key elements of electrode switching. In the first two lines of code in FIG. 7, the eight RAB ports and the five RAA ports of the PIC 16F84 are set to output. Combinations of these thirteen outputs may then be switched so as to activate the optothyristors 40, 92, 94 controlling electrode elements 12,14. The code lines in the form of “Poke A, B” signify which of the inner and outer electrodes are to be energized. The code lines in the form of “Pause X” signify the time duration between activations of electrode elements 12,14 to fully turn off the optothyristors and accommodate the response time of the optothyristors. The PIC Microprocessor 20 may be operated up to 20 MHz. In one embodiment, processor 20 is operated at 4 MHz with a clock frequency of 10 MHz. In this case, “Pause 1 ” corresponds to a pause of 400 microseconds, “Pause 3” corresponds to a pause of 1200 microseconds (3×400 microseconds), etc.

When skin penetration occurs, the possible tendency for the TENS signal to “short circuit” through the needle track in the epidermis can be accommodated by switching the signals between the inner electrodes and/or the outer electrodes. Thus, the TENS current may be relatively increased within the poorly conducting outer layers of the skin (specifically the epidermis) as compared to the more dominant usual route whereby current tracks through sweat glands or hair follicles down to the lower electrical resistance areas of the dermis.

Increasing electrode area reduces the resistance between the electrode and the sub-dermal tissue (which in absolute terms with normal sized electrodes is several orders of magnitude lower than a purely epidermal pathway resistance between electrodes). This is because far more sweat glands or hair follicles are covered by the electrode surface. As inter-electrode distance approaches or is less than twice epidermal thickness, conduction along the epidermal tissue becomes significant in proportion to the total current flow, and electrode area reduction further improves this efficiency at the expense of electrode effectiveness. However, the applied voltage needed to cause this current to flow increases. Thus, a balance may be struck between area, voltage and inter-electrode gap such that both epidermal and dermal nerves are adequately exposed to the TENS signal.

The present invention provides the un-myelinated “C” nerve fiber endings that lie within the epidermis and are part of the pain sensing mechanism with better exposure to the depolarizing current. In the dermis, it is believed that other nerve fiber types, particularly the A-delta fibers, may also conduct pain signals. There are multiple low resistance routes for the current to take both within and below the dermis that avoid flow around nerve endings, limiting the distance between electrodes and electrode surface areas, and increasing the electrical potential or gradient in a small area. This both increases the probability of a nerve fiber in this region being depolarized or hyper-polarized and decreases the probability of current tracking into deeper tissues and causing side effects such as muscle stimulation. Further, limitation of electrode area decreases electrode-to-muscle capacitance, thereby reducing signal conduction by capacitive coupling to this tissue and so reducing muscle stimulation.

In known applications of TENS, acclimatization to the TENS current occurs with respect to discomfort caused by the current. This tolerance to the TENS current evolves in the patient over a short period of time, particularly with a continuous pulse signal as opposed to bursts of pulses at a low frequency. Thus, if the signal applied to the electrodes is built up slowly, such as over the course of a few seconds, significantly higher signal strengths may be applied without causing significant discomfort. In the apparatus of the present invention, electrical circuitry is so arranged that the TENS signal(s) builds up in intensity over a few seconds. In addition, circuitry is provided that reduces the pulse width of the signal over a few seconds, again allowing a higher voltage to be applied without discomfort. This combination of electrode surface limitation, inter-electrode gap limitation, signal strength buildup, pulse width control, outer electrode use and interplay with the inner electrodes as well as the timing and pattern (both in time and locality) of TENS signal application allow the use of an intense local TENS effect. This results in blocking and/or distorting the usual pain signal formation and pattern of firing of the local nerves in the area of skin penetration. The overall effect is to cause miscommunication and misinterpretation of the pain signals at higher levels, in effect blocking or drastically reducing the sensation of pain experienced by the local tissue trauma. Each of these elements contributes in part to the overall effect, resulting in a relevant level of efficacy and comfort.

There are a variety of nerve fiber parameters, frequencies and waveforms that may be used in conjunction with the present invention. The possible frequency of action potentials of nerve fibers conducting pain signals may be measured or estimated, and from this, favorable blocking signal parameters may be determined. Such information is available to a degree in standard texts of physiology. However, it is likely that there are both a range of fiber types and dimensions as well as their possible branching and networking arrangements that need to be addressed by the TENS signal. In some circumstances, as nerve branches coalesce into the main trunk, for example, the frequency of transmitted impulses decline, in effect acting as a low pass filter. Further, it is known that there is not a simple one to one relationship between frequency of discharge, number of nerves involved or of nerve type and the perceived sensation of pain severity. These observations are consistent with a system with redundancy as described above. Electrically, the skin acts as both a resistive and a capacitive barrier in parallel, thus an increase in frequency naturally results in lower impedance to current flow. Higher frequencies tend to direct the signal to muscle which itself has a surface area capacitive quotient that is higher than that of nerve by a factor of ten or more. Moreover, muscle has more total surface area than nerve. Thus, the strategy taken here is to present a sweep of pulses and amplitudes that fit a range of possibilities. TENS is usually applied as a core depolarizing pulse of the order of 50 to 200 micro-seconds long repetitively applied in a train, which may be broken up into blocks or bursts of pulses. It has been found that a shorter pulse width of 30 to 150 microseconds has an adequate level of effectiveness, though longer pulses are common in studies on nerve and are more effective in nerve stimulation. Applying these various signal forms through the electrodes of the present invention produces the desired effect. If a prolonged exposure is desired, such as with a wound dressing, modulation of the plateau level of signal with a time constant of the order of a few tenths to hundredths of a second may help ensure continuing efficacy.

It is also possible within the scope of the present invention to apply physical vibration to the needle during the injection. The perception of the severity of pain is related amongst other things to the force or energy of injury (Differential ability of human cutaneous nociceptors to signal mechanical pain and to produce vasodilatation. Koltzenburg, Handwerker, J Neurosci 1994 Mar; 14(3 Pt 2):1756-65). Vibration lowers the resistance to a needle entering the skin in the same manner that wiggling the fingers in sand allows them to penetrate the sand more easily. In addition, the provision of a vibration during the time of injection adds another distracting element to the nervous system. Thus, a vibration generating device may be included in order to enhance the effectiveness of the present invention.

In general, an inner area proximal to the site of skin breach has an applied pattern of TENS activity of a regular or, more usually, an irregular spacing between pulses, both in time and the specific route of electrical discharge. The time interval between individual pulses in this inner electrode area may be generally a few milliseconds (although it may be longer), whereas full coverage of the anatomy of this inner electrode area by a TENS signal takes a few tenths or, more usually, a hundredth of a second or less.

A second area, more peripheral to the site of injection or tissue damage, has a pulse pattern which may be regular or, more usually, irregular with inter-pulse pauses that may vary up to a few tenths of a second. This latter area is less demanding in the specifications required of TENS application, excepting the pulse interval pattern. The latter pattern of pulses may be added to or superimposed on the inner electrode elements in addition to their own blocking pattern, but a higher intensity is required where the two signals overlap. The effect of the outer element signal pattern, or its overlay on the inner electrode elements, is such that a sensation of irregular or patterned buzzing or prickling is noticed by the patient. This sharp irregular sensation makes it difficult or impossible to recognize the timing of the sharp sensation that is produced when a sharp instrument (needle) contacts the skin. This sensation is one of the key elements in the normal perception of pain during an injection and its disruption contributes to the efficacy of this system.

The two electrode areas may intermittently have pulses applied between individual electrode elements of each area, although for the most part they are independently active, and thus may both be energized as separate circuits at the same time. When both areas are energized at the same time, in effect, the overall signal strength may be double at that instant.

The known designs of signal application electrodes used for TENS, as well as the type of TENS generating and control units, are not appropriate for applying signals to two separate electrode areas as in the method of the present invention. Rather, known electrode designs are better adapted to wider and deeper signal penetration utilizing electrodes of a larger area and with fewer electrode elements capable of being exposed to different signals.

The electrodes of the present invention mat be arranged such that the gaps or spacing between opposite conducting electrode edges in the region of skin puncture are kept within an order of magnitude of the thickness of skin (epidermal thickness is usually about 0.2 millimeter). Thus, the electrode-to-electrode elements gap in the region of and adjacent to the skin puncture may be of the order of a millimeter or so, except where needle dimensions allow for less, or require a greater gap, such as for an intra-vascular cannula insertion. In the situation where a larger space is required for the injection (for example a cannula insertion), the inter-electrode gap is kept to a minimum by employing additional electrode elements wherever possible within the inner encircling ring that circumscribes the area of skin puncture. This inner electrode arrangement affords more anatomically controlled and exact local TENS exposure of the epidermal and dermal nerve fibers, particularly C fibers which have terminals located in the epidermis as well as in deeper layers. In addition, the deeper sited nerve fibers may be interacted with by these electrodes and the more widely spaced electrode combinations, switching of the TENS signal between individual and multiple electrode surfaces of the overall electrode matrix in rapid succession.

In addition to the relatively small gap between electrode elements, the surface area of individual electrode elements may be relatively small (2-5 square millimeters or less), particularly for those electrode most proximal or nearest to the injection site. This further aids the localization of current flow, reducing the tendency for deeper and wider tissue stimulation and muscle stimulation that occurs with known electrode arrangements with larger surface areas active at any one instant. The total area of tissue exposure may be increased or widened by switching activity in electrode pairs in a rapid sequential manner across the total electrode matrix area. By so doing, the area active at any one instant is small, yet coverage of the total area requiring TENS exposure is affected.

The TENS signals are applied across this array of electrodes and may be varied in polarity, voltage, frequency, wave form and timing of application. Different anatomical locations on the body may require different settings for maximum effect and comfort. Within the locale of the electrode matrix, different electrode pairs or combinations may be concurrently targeted for either nerve blockade or counter irritation nerve stimulation, thereby causing a distraction signal to the patient.

In areas less proximal to the site of skin puncture, the TENS application may provide counter irritation or a distracting signal, and may possibly blockade the pain signals. The peripheral (distracting) signal also may allow the centrally located electrode surfaces to deliver a higher strength of signal than would otherwise be comfortable, to a degree masking the more tonic sensation created by the signals applied to these electrode elements. The outer element function requires a less exacting specification for current application depth, and therefore electrode size and geometry may be larger and more separated.

The TENS signal waveform applied may be a standard repetitive uni- or bipolar pulse (either in a prolonged series or in bursts). More complex waveforms may be used, such as described in U.S. Pat. No. 4,723,552. This latter waveform may allow for the tuning of the TENS current to the characteristics of the targeted nerve and nerve endings, reducing the tendency of spread and allowing a lower power TENS signal to be effective. However, with the arrangement of the present invention, a simple unidirectional pulse may be effective, when applied in the fashion described below, particularly noting the frequency and irregularity of pulse intervals between pulses as they are applied through the two electrode areas.

The provision of a concurrent physical vibration to the device and/or skin both reduces the direct force and energy required for needle penetration (which is related to the perception of pain severity) as well as providing an additional distracting stimulus to the patient and consequent masking of pain perception. Using the same principles of an inner and outer electrode element matrix, the electrode may be fashioned into a wound dressing, or the electrode array may be fashioned so as to allow biopsy or other surgical device use. In these cases and uses, the smaller and more closely spaced electrode elements are most proximal to the area of tissue injury. Further, as a consequence of vascular smooth muscle contraction resulting either directly or through reflex to the local application of an intense TENS current, hemorrhage may be diminished or eliminated.

The present invention provides a more complete elimination of coherent pain signaling, as well as an improved method of disorganizing and fragmenting unblocked pain signals that are induced upon tissue injury. In addition, the present invention may be adapted so as to be used during other procedures where the skin is breached, such as the insertion of an intravascular cannula or other medical device such as a pharmaceutical slow release capsule. Further, the present invention may be modified such that the electrode surface may be incorporated into a surgical/medical dressing allowing for the application of the electrical signals, thereby facilitating hemorrhage control and analgesia. Additionally, the present invention may be modified such that the electrode surface may be inserted into wound for preventing or reducing hemprrhage. An additional vibration element may allow soothing or analgesic effect on a wound.

This invention provides a method and apparatus for reducing or eliminating pain on skin breach, such as during a therapeutic injection, by the application of TENS with or without a co-stimulus of vibration before, during and after an injection. The method may also be applicable for superficial pain relief or hemorrhage as may be useful in the management of a surgical or other wounds. The beneficial effect may be achieved through the use of an electrode array that meets certain defined specifications in the size and arrangement of electrode elements, in association with a control unit that switches TENS signals between these various electrode elements in a specified manner. Thus, the locality of the TENS current can be controlled and differing nerve fibers can be independently targeted. The pattern of electrode activity is such that the total exposure area may have different signal patterns applied within different regions of this total exposure area.

Reducing general communication of pain signals and adding disinformation into the communication pathways is more likely to be effective with poorly interpretable or uninterpretable signals that reach the central nervous system from the periphery. Thus, the present invention improves local delivery of TENS to a specific area and adds signals that disrupt interpretation of remaining signals. More particularly, counterfeit or noise signals may be added to those nerves still operating at a biologically adequate level. Thus, the central nervous system does not receive enough consistent information to correctly register the signals of skin breach and tissue injury. Specifically, the electrode layout and switching activity between electrode pairs increases local TENS exposure proximal to the injection site, affording an optimized level of local blockade, incomplete as it is liable to be. Another pain-mitigating factor is that the irregular or difficult to interpret timing pattern of strong pulses applied to the outer elements or in the background of more frequent (blocking) pulses makes it difficult for the patient to recognize the timing and sharpness of skin penetration. Thus, the normal neuronal activity of skin breach is blocked and masked by the background of disrupting signals and information.

Were a TENS signal to be applied by a needle as one pole of the apparatus of the present invention, the same problems of the prior art would arise unless opposite electrodes were applied with a gap between them of about a millimeter and there were multiple conduction pathways. By reducing electrode contact area and inter-electrode gap, the present invention controls the anatomy of current flow, in particular where the skin acts as a dielectric conductor and breaks down its resistance under the electric field. Thus, the present invention provides a surety of anatomical nerve or nerve branch targeting, which is impossible with the known TENS electrode designs. The present invention may utilize two distinct electrode designs for superficial nerve blockade electrodes and the surrounding TENS counter irritation electrodes.

There are several advantages of the present invention. The present invention makes use of TENS or other electrical current to treat pain associated with a localized injury, such as from the injection of a needle. Second, the present invention includes the use of electrodes that are more numerous and smaller in operable surface area than the electrodes used in known TENS techniques. Smaller electrodes have the advantage of being able to be placed closer to the needle, and are better suited to provide a current that is localized in the area of the injection. Smaller electrodes also reduce capacitive coupling and thus reduce the amount of current lost to the muscles. Third, the voltages applied to the electrodes in the present invention are much greater than the voltages applied in traditional TENS. Voltages up to approximately 1000 volts may be applied in the present invention, while traditional TENS applied voltages of less than 100 volts. Fourth, in the present invention, TENS is applied for a much shorter length of time, such as less than ten seconds.

While this invention has been described as having an exemplary design, the present invention may be further modified within the spirit and scope of this disclosure. This application is therefore intended to cover any variations, uses, or adaptations of the invention using its general principles.

1. An apparatus for reducing pain or hemorrhage, the apparatus comprising:
a current generating device configured to generate an electrical output Trans Epithelial Nerve Stimulating (TENS) current; and
an array of electrodes electrically coupled to the current generating device and configured to be placed around an injection location on the skin of a patient, each of said electrodes has a surface area of less than five square millimeters.

2. The apparatus of claim 1, wherein each said electrode is no more than two millimeters away from an adjacent one of said electrodes.

3. The apparatus of claim 1, wherein the injection location is less than one millimeter from at least one of said electrodes.

4. The apparatus of claim 1 wherein the array of electrodes comprises a first array of electrodes, said apparatus further comprising a second array of electrodes, the current generating device being configured to generate a first type of the TENS current for the first array of electrodes and a second type of the TENS current for the second array of electrodes.

5. The apparatus of claim 4 wherein said first array of electrodes is adapted to be disposed proximal to and circumscribe the injection location.

6. The apparatus of claim 4, wherein each said electrode of said first array is spaced less than two millimeters away from an adjacent electrode of said second array.

7. The apparatus of claim 4 wherein said electrodes of said second array of electrodes substantially surround said electrodes of said first array.

8. The apparatus of claim 4 wherein each of the electrodes of the second array is controlled independently of each of the electrodes of the second array.

9. An apparatus for reducing pain or hemorrhage at an injection location on the skin of a patient, the device comprising:
a current generating device configured to generate first and second types of electrical output Trans Epithelial Nerve Stimulating (TENS) current, the first type of TENS current having a frequency greater than about 15 Hz, the second type of TENS current having a frequency less than about 15 Hz;
an array of electrodes electrically coupled to the current generating device and configured to be placed around the location on the skin of the patient, the electrodes including a first group and a second group, the first group being configured to be disposed closer than the second group to the injection location on the skin of the patient; and
a current applying device configured to:
apply the first type of TENS current to the first group of electrodes; and
apply the second type of TENS current to the second group of electrodes.

10. The apparatus of claim 9 wherein the first type of TENS current has a frequency of approximately between 25 and 200 Hz, and the second type of TENS current has a frequency of less than about 10 Hz.

11. The apparatus of claim 9 wherein the current applying device is capable of activating the first group of electrodes in a pattern such that the injection location is effectively encircled by the TENS current.

12. The apparatus of claim 11 wherein the TENS current is applied in an encircling pattern having a plurality of pulses of varying power and varying duration.

13. The apparatus of claim 12 wherein the encircling pattern of the first group of electrodes is different from the encircling pattern of the second group of electrodes.

14. The apparatus of claim 9 wherein said current applying device is configured to apply a voltage of greater than 300 volts to at least one of the first group of electrodes and the second group of electrodes.

15. The apparatus of claim 9 wherein the first type of TENS current is controlled independently of the second type of TENS current.

16. A method of reducing pain or hemorrhage of a therapeutic injection or a body tissue sampling, the method comprising the steps of:
placing an array of electrodes on the skin of a patient;
applying electrical output Trans Epithelial Nerve Stimulating (TENS) current to the electrodes; and
breaching the skin of the patient at a location within the array of electrodes during said applying step.

17. The method of claim 16 wherein a duration of said applying step is less than 30 seconds.

18. The method of claim 16 wherein the array of electrodes includes a first group and a second group, the location of the skin breach being closer to the first group than to the second group.

19. The method of claim 18 wherein the applying step includes applying a different type of TENS current to the first group of electrodes than to the second group of electrodes.

20. The method of claim 19 wherein the second group of electrodes encircles the first group of electrodes.

21. A method of reducing pain or hemorrhage, the method comprising the steps of:
placing an array of electrodes into a patient's wound;
applying electrical current to the array of electrodes.

22. The method of claim 21 further comprising breaching a patient's skin at a location within the array of electrodes during said applying step.

23. The method of claim 21 wherein a duration of said applying step is less than 30 seconds.

24. The method of claim 21 wherein the array of electrodes includes a first group and a second group, the location of the skin breach being closer to the first group than to the second group.

25. The method of claim 24 wherein the applying step includes applying a different type of current to the first group of electrodes than to the second group of electrodes.

26. The method of claim 25 wherein the second group of electrodes encircles the first group of electrodes.
